(19) United States
(12) Patent Application Publication
(10) Pub. No.: US 2013/0138003 A1
(43) Pub. Date: May 30, 2013

(54) METHOD, APPARATUS AND COMPUTER PROGRAM PRODUCT FOR MONITORING CLINICAL STATE OF A SUBJECT

(71) Applicant: Mikko Kaski, Espoo (FI)

(72) Inventor: Mikko Kaski, Espoo (FI)

(73) Assignee: GENERAL ELECTRIC COMPANY, Schenectady, NY (US)

(21) Appl. No.: 13/307,477

(22) Filed: Nov. 30, 2011

(51) Int. Cl. A61B 5/0402

(52) U.S. Cl. 600/509

(57) ABSTRACT

A method, apparatus, and computer program product for monitoring clinical state of a subject are disclosed. To provide a mechanism that allows perception of the clinical state of the subject easily and without expertise, an evolution measure is determined for each of a plurality of physiological channel signals acquired from a subject, thereby to obtain a corresponding plurality of channel-specific evolution measures, wherein each channel-specific evolution measure is indicative of development in respective channel signal. Each channel-specific evolution measure is mapped to a plot color, thereby to obtain a channel-specific plot color for each of the plurality of physiological channel signals, wherein the determining and mapping are carried out in successive time windows, thereby to obtain a corresponding plurality of channel-specific plot color sequences, which are presented to a user as an indication of the clinical state of the subject.

### BACKGROUND OF THE INVENTION

This disclosure relates generally to patient monitors. More particularly, the present invention relates to monitoring of physiological signals, especially electrocardiograms, to monitor the clinical state of a subject.

Patient monitors are electronic devices designed to display physiological information about a subject. Electrocardiogram (ECG), electroencephalogram (EEG), plethysmographic signals, and signals related to blood pressure, temperature, and respiration represent typical physiological information contained in full-size patient monitors. Patient monitors are typically also furnished with alarming functionality to alert the nursing staff when a vital sign or physiological parameter of a patient exceeds or drops below a preset limit. Alarms are normally both audible and visual effects aiming to alert the staff to a life-threatening condition or to another event considered vital. In most monitors, the alarm limits may be defined by the user, since the limits typically depend on patient etiology, age, gender, medication, and various other subjective factors. Each specific physiological parameter, such as heart rate or blood pressure, may also be assigned more than one alarm limit. Furthermore, there is a lot of data available for caregivers in a patient monitor.

However, alarms are often activated in a phase where the situation is already critical and the vast amount of data available in a monitor is typically in a form that needs further processing and additional analyses to be useful. Current patient monitors cannot process this data quickly enough to a form that would be directly useful for caregivers to take an action in advance in order to avoid a critical or severe situation.

For recording an electrocardiogram, electrocardiographic leads are used at specified locations of the subject for recording ECG waveforms. In typical clinical practice, 12 leads are used to the record the ECG. However, the number of leads used may vary. Each lead records a waveform representing the electrical activity generated by the heart cardiac cycle by cycle and together the lead recordings provide spatial information about the heart's electrical activity.

A normal cardiac cycle includes contractions of the atrial muscles, which are activated by the autonomic sinoatrial node (SA node), also called the sinus node. An electrophysiologic (EP) signal generated by the SA node spreads in the right and left atrium leading to their contraction. The EP signal further reaches the atrioventricular node (AV node) situated between the atria and the ventricles. The AV node delays the EP signal, giving the atria time to contract completely before the ventricles are stimulated. After the delay in the AV node, the EP signal spreads to the ventricles via the fibers of the His-Purkinje system leading to the contraction of the ventricles. After the contraction, the atria are relaxed and filled by blood coming from venous return. The entire cardiac cycle is the combination of atrial and ventricular contraction, i.e. depolarization, and their relaxation, i.e. repolarization.

In this connection, reference is made to FIG. 1, which illustrates one cycle of a normal ECG signal. As is commonly known, and also shown in the figure, the waves of the ECG signal (i.e. the depolarization and repolarization events in the heart) are named alphabetically from P to U. Normal ECG signal shows each phase of the cardiac cycle: the P wave represents the systole of the atria, the QRS wave represents the systole of ventricles, and T wave represents the repolarization of the ventricles. Modern ECG devices use digital signal processing to analyze the shape and consistency of these waveforms, and also the durations between the waveforms.

In clinical environment, the first decision needed normally in view of an ECG is whether or not the ECG is normal. For a trained physician the examination of an ECG in this respect is more or less a routine task. However, in a clinical environment a trained physician is not always available for ECG interpretation and nurses are not normally trained to analyze the ECG waveforms. Current patient monitors lack intelligence to evaluate the ECG waveforms in this respect and cannot therefore assist the nurses in the decision-making. Therefore, physicians may be called in unnecessarily or abnormalities in cardiac function may remain unnoticed before a trained physician is available for ECG analysis.

In terms of efficient care, it is also important that the personnel could detect abnormal cardiac function and contact a trained physician quickly after the onset of cardiac dysfunction and that as relevant and detailed information as possible could be given to the physician about the nature of the detected event. One reason for this is that some signs may be early and subtle warnings of a more severe event to come and some signs may indicate, for example, that a more severe event may have occurred locally in the heart but cannot be detected properly due to a reason, such as limited number of ECG leads. The occurrences of different heart attack types called STEMI (ST segment Elevation Myocardial Infarction) and NSTEMI (Non-ST segment Elevation Myocardial Infarction) are related to each other in these respects. STEMI, which is a severe type of heart attack, is caused by a prolonged period of blocked blood supply and manifested by an elevation in the level of the ST segment of the QRS wave. In NSTEMI, i.e. in the “milder” type of heart attack, the blood clot only partly occludes the artery and no elevation in the ST segment of the ECG may be present, which makes an NSTEMI event more difficult to detect. However, an NSTEMI event may develop to a STEMI event and detected NSTEMI events may indicate that a STEMI event has occurred or is about to occur locally. NSTEMI alone may also be an important indication of a severe condition, especially in patients with prior cardiac damage.

However, due to the limited ability of the nursing personnel to interpret the clinical state of the patient, the above goals are difficult to achieve and therefore appropriate and efficient treatment may be delayed until a trained physician is available at bedside.

### BRIEF DESCRIPTION OF THE INVENTION

The above-mentioned problems are addressed herein which will be comprehended from the following specification. In the disclosed monitoring system, an evolution measure indicative of the development of a physiological signal is determined on several measurement channels. The channel-specific evolution measures are mapped or converted to display colors and the colors may be presented on channel-specific zones of an evolution plot that serves as a display element that visualizes the clinical state of the subject in multiple dimensions to facilitate the interpretation of the changing state of the patient. By looking at the evolution plot, the nursing staff may easily get an idea of the extent, location, severity, and temporal duration of an abnormal event and can give valuable initial information to the expert contacted.

In an embodiment, a method for monitoring clinical state of a subject comprises determining an evolution measure for each of a plurality of physiological channel signals acquired from a subject, thereby to obtain a corresponding plurality of channel-specific evolution measures, wherein each channel-specific evolution measure is indicative of development in respective channel signal. The method also comprises mapping each channel-specific evolution measure to a plot color, thereby to obtain a channel-specific plot color for each of the plurality of physiological channel signals, wherein the determining and mapping are carried out in successive time windows, thereby to obtain a corresponding plurality of channel-specific plot color sequences. The method further comprises presenting the corresponding plurality of channel-specific plot color sequences to a user as an indication of clinical state of the subject.

In another embodiment, an apparatus for monitoring clinical state of a subject comprises an evolution determination unit configured to determine an evolution measure for each of a plurality of physiological channel signals acquired from a subject, thereby to obtain a corresponding plurality of channel-specific evolution measures, wherein each channel-specific evolution measure is indicative of signal development in respective channel signal. The apparatus also comprises a mapping unit configured to map each evolution measure to a plot color, thereby to obtain a channel-specific plot color for each of the plurality of physiological channel signals, wherein the evolution determination unit and the mapping unit are configured to operate in successive time windows, thereby to obtain a corresponding plurality of channel-specific plot color sequences. The apparatus further comprises a presentation unit configured to present the corresponding plurality of plot color sequences to a user as an indication of clinical state of the subject.

In a still further embodiment, a computer program product for monitoring clinical state of a subject comprises a first program product portion adapted to determine an evolution measure for each of a plurality of physiological channel signals acquired from a subject, thereby to obtain a corresponding plurality of channel-specific evolution measures, wherein each channel-specific evolution measure is indicative of signal development in respective channel signal. The computer program product also comprises a second program product portion adapted to map each evolution measure to a plot color, thereby to obtain a channel-specific plot color for each of the plurality of physiological channel signals, wherein the first and second program product portions are adapted to operate in successive time windows, thereby to obtain a corresponding plurality of channel-specific plot color sequences. The computer program product further comprises a third program product portion adapted to present the corresponding plurality of plot color sequences to a user as an indication of clinical state of the subject.

Various other features, objects, and advantages of the invention will be made apparent to those skilled in the art from the following detailed description and accompanying drawings.

### BRIEF DESCRIPTION OF THE DRAWINGS

FIG. 1 illustrates one cycle of a normal ECG signal;

FIG. 2 is a flow diagram illustrating an embodiment of the operation of a physiological monitor in terms of the visualization of the clinical state of a subject;

FIG. 3 illustrates one embodiment of the evolution plot presented to the user;

FIG. 4 illustrates typical changes in the ECG cycle when myocardial infarction develops;

FIGS. 5 to 7 illustrate examples of views presented to the user in connection with an acute myocardial infarction;

FIG. 8 illustrates an embodiment of an ECG monitoring apparatus/system; and

FIG. 9 illustrates the operational entities of the ECG monitoring apparatus/system in terms of the presentation of the clinical state of a subject.

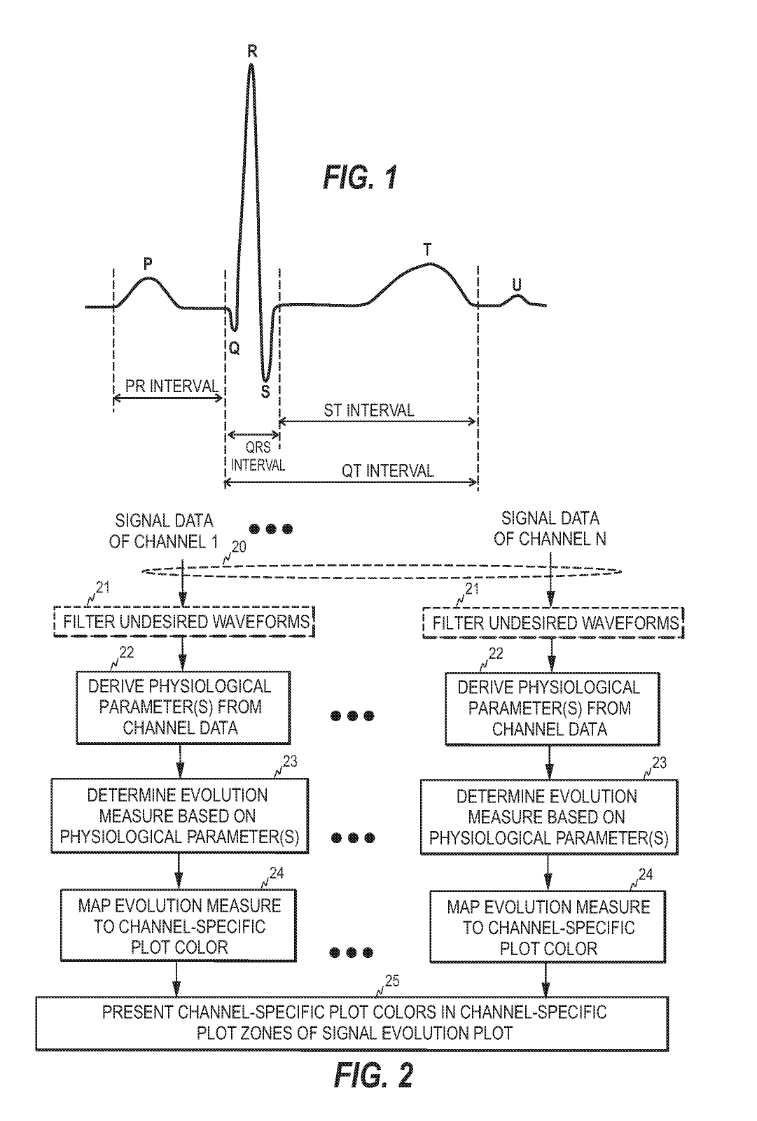

### DETAILED DESCRIPTION OF THE INVENTION

FIG. 2 illustrates an embodiment of the monitoring method in terms of the visualization. Physiological signal data is acquired from a subject on a plurality of measurement channels 20. Depending on the application, an optional filtering step 21 may be used prior to the actual signal processing steps to filter out undesired waveforms. On each channel, the following steps 22-24 may be carried out. One or more physiological parameters are first derived from the channel data (steps 22). Based on the physiological parameter(s), an evolution measure is then determined in each step 23. This determination may be based on reference data determined and stored in advance for the parameter(s) of the subject or for the evolution measure itself. In other words, the evolution measure is typically a variable that is indicative of the development that has occurred in the channel signal in relation to a reference state, which is typically the state regarded as the normal state of the subject. Consequently, the evolution measure reflects the severity of the subject's state with respect to the reference state. If only one physiological parameter is derived, the evolution measure may be derived from the difference of the current and reference values of the parameter. If multiple physiological parameters are derived on each channel, the evolution measure may be an aggregate value that may be calculated, for example, as a weighted sum of the differences or absolute differences. The reference data may also comprise a reference waveform, such as reference QRS wave whose morphology defines the reference state with which the current state, i.e. the current waveform, is compared.

The evolution measure of each channel is then mapped or converted to a plot color (steps 24) that depends on the current value of the evolution measure. It is assumed below that evolution measure values that indicate no or insignificant changes with respect to the reference state are mapped to green. However, the reference state may also be mapped to white or any other appropriate color.

An evolution plot is then produced from the channel-specific plot colors and displayed to the user (step 25). In the evolution plot, a zone or section is assigned to each channel. The above steps are carried out for the successive/consecutive time segments/windows of the channel signals, thereby to obtain a plot color for each time segment of each plot zone. In ECG monitoring, the time segment may correspond to one cardiac cycle of the subject, for example.

The color scale used may reflect the severity of the change occurring in the subject. The evolution plot serves as a display element that visualizes the state of a subject in multiple dimensions (channels involved (i.e. locality)/duration time/severity) to facilitate the interpretation of the changes that occur and to provide an easily comprehensible image of the actual state of the subject.

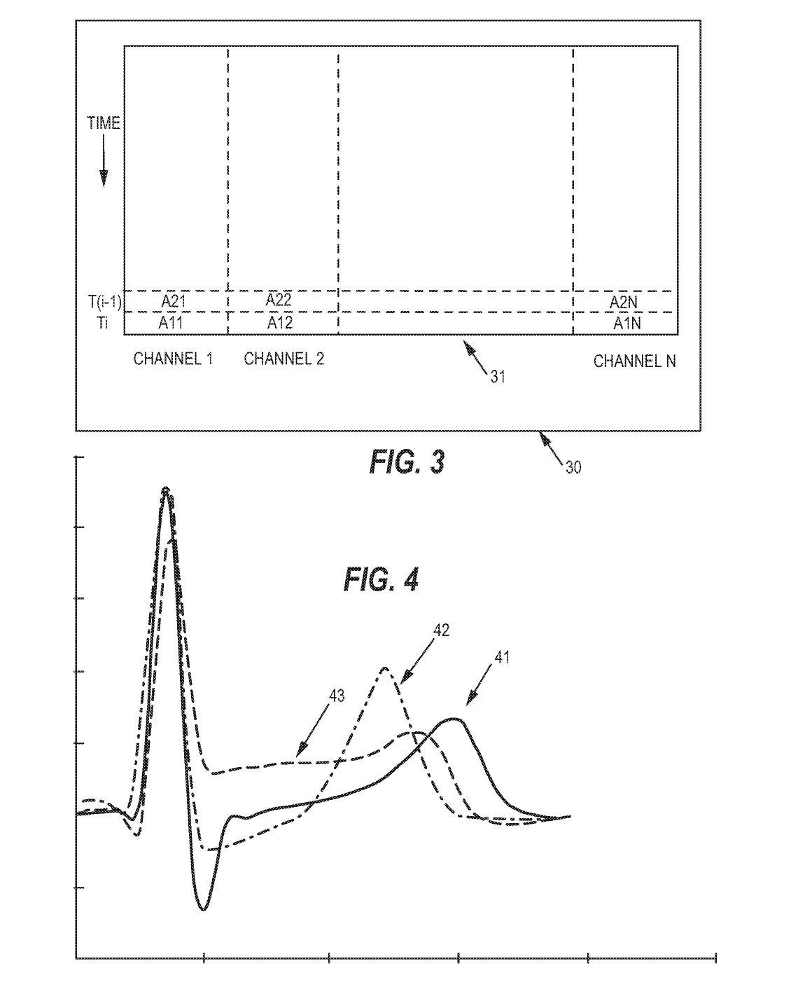

FIG. 3 illustrates an example of a user display window 30 comprising an evolution plot 31 in which the vertical axis represents time and the horizontal axis is divided between the channels. Each channel corresponds to a “column” that forms a channel-specific plot zone in which the plot color sequence of that channel is displayed. Consequently, the color of the area denoted with A11 corresponds to the evolution measure of channel 1 in current time window Ti, the color of the area denoted with A12 to the evolution measure of channel 2 in current time window Ti, etc. The color of the area denoted with A21 in turn corresponds to the evolution measure of channel 1 in previous time window T(i−1) and the color of the area denoted with A22 to the evolution measure of channel 2 in the previous time window T(i−1), etc. In the figure, the dashed lines are used for illustrative purposes; no lines may be displayed in real user view. The content of the display window may also be rotated with respect to the orientation shown in FIG. 3, i.e. the horizontal axis may represent time and the vertical axis may be divided between the channels.

If all evolution measures are in normal value range, the evolution plot is plain, for example plain green, if green is used to indicate normal state. However, if significant changes start to occur on a channel, the plot color of this channel starts to change according to the change in the evolution measure of the channel. The greater the deviation from normal values, the more pronounced the change in the color. For example, a slight deviation from normal may be indicated with light blue, and the blue shade may become darker as the severity, indicated by the evolution measure, increases.

The evolution plot 31 provides kind of 3D representation of the state of the subject. If abnormalities occur, the horizontal (or vertical) axis indicates the channel(s) on which the disorders occur (locality), the vertical (or horizontal) axis indicates the duration of the disorder, and the third axis (color) indicates the severity of the disorder. Consequently, when a nurse notices that a plain evolution plot starts to change (s)he can provide valuable information about the state of the subject.

Although it is possible to utilize the above solution for various physiological signals, the monitoring mechanism is particularly useful in connection with ECG monitoring. The examples below assume that an ECG of a subject is monitored.

As mentioned above, the more severe form of myocardial infarction, called STEMI, is manifested by an elevation in the ST segment level. However, decreased or elevated T waves may be early indications of an infarction, even 30 minutes before changes in the ST level can be detected. FIG. 4 illustrates an example of how the QRS complex of a subject may change when a STEMI event is developed. Curve 41 represents a QRS complex considered as normal for the subject, in QRS complex 42 the T wave is elevated prior to a STEMI event, and in QRS complex 43 the ST level is elevated (STEMI). Consequently, T wave amplitude and ST level are typical parameters that may be determined in steps 22 of FIG. 2. Other physiological parameters that may be determined include T wave integral, QRS amplitude, Q wave amplitude, QT time, T wave shape (like symmetry and/or flatness), heart rate, and T wave short term variation.

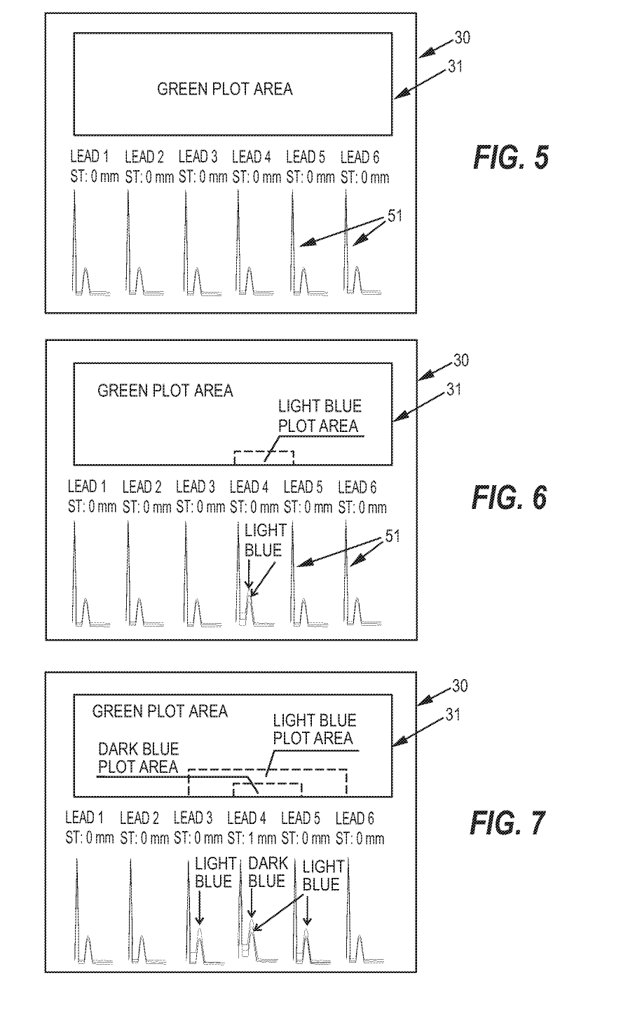

FIGS. 5 to 7 illustrate how the evolution plot may visualize an acute myocardial infarction. It is assumed here that T wave amplitude and ST segment level are measured from each QRS cycle on each channel. In the examples of FIGS. 5 to 7, the waveforms 51 of the latest QRS complexes are presented on top of each other below the respective plot zone of the evolution plot. Furthermore, the ST level values are presented as millimeter values above the QRS complexes (in standard ECG measurement, 10 mm corresponds to 1 mV).

In FIG. 5, the evolution plot 31 is plain green indicating that the state of the subject is normal. That is, the QRS wave does not deviate significantly from its reference wave 41. Consequently, the QRS waveforms shown beneath the evolution plot are also green. In the course of time, the first signs of an acute myocardial infarction start to appear as changes in the T wave morphology of the QRS waveform. The changes are in this example concentrated around lead 4. As the T wave morphology of lead 4 starts to change, the evolution measure of the lead increases and the color of the plot zone of lead 4 starts to change. In this example, the green color changes to light blue when the evolution measure crosses the limit of normal range. The color of the QRS curve 51 displayed may change similarly, as is shown in FIG. 6. Over time the abnormalities become more evident and the evolution measure deviates more from the normal range. The greater the deviation, the deeper the shade of blue that can be seen in the respective plot zone, cf. FIG. 7.

The colors may also indicate the direction of the change. For example, an increase in the amplitude of T wave may be indicated by red, while a decrease or inversion in the T wave may be indicated by blue.

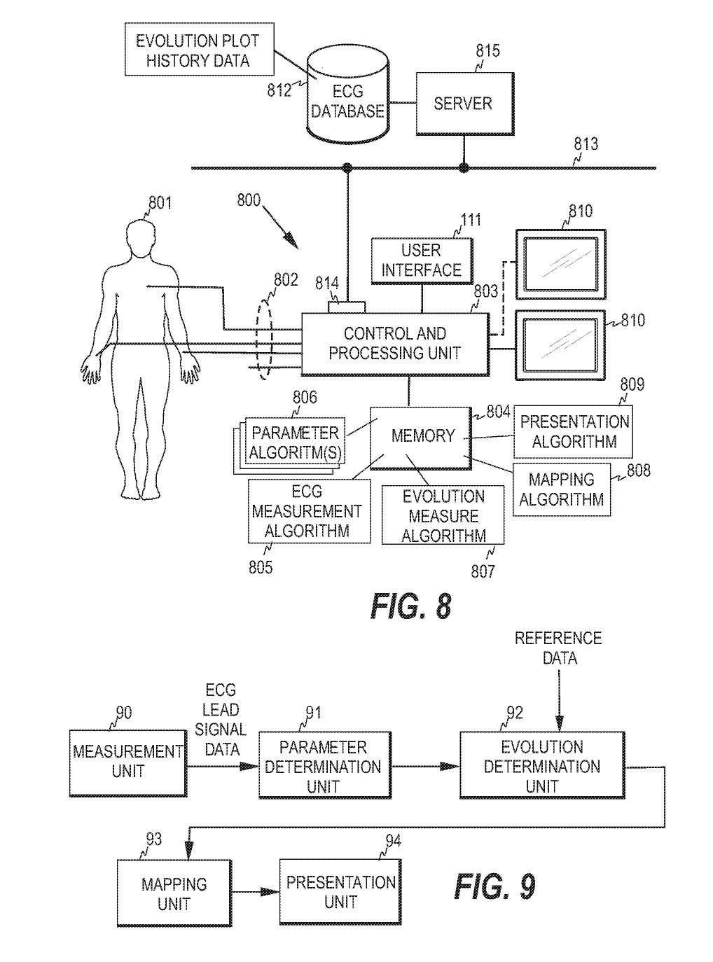

FIG. 8 illustrates one embodiment of a monitoring apparatus/system 800 for monitoring a subject 801. A monitoring apparatus/system normally acquires a plurality of physiological signals 802 from the subject, where one physiological signal corresponds to one measurement channel. The physiological signals may typically comprise several types of signals, such as ECG, EEG, blood pressure, respiration, and plethysmographic signals. Based on the raw real-time physiological signal data obtained from the subject, a plurality of physiological parameters may be determined. A physiological parameter here refers to a variable calculated from the waveform data of one or more channel signals acquired from the subject. Thus, channels may be grouped and an evolution measure may be calculated for the group rather than for an individual channel. Therefore, the term channel may here refer to a single channel or to a group of sub-channels forming the channel and the term channel-specific may refer to something that concerns an individual channel or a group of sub-channels forming the channel. The physiological parameter may also represent a waveform signal value determined over a predefined period of time, although the physiological parameter is typically a distinct parameter derived from one or more measurement channels. Each signal parameter may be assigned one or more alarm limits to alert the nursing staff when the parameter reaches or crosses the alarm limit.

The physiological channel signals 802 acquired from the subject 801 are supplied to a control and processing unit 803 through a pre-processing stage (not shown) comprising typically an input amplifier and a filter, for example. The control and processing unit converts the signals into digitized format for each measurement channel. The digitized signal data may then be stored in the memory 804 of the control and processing unit. The memory may also store the reference data that define the normal state for each physiological parameter. The same parameter may have different reference values on different channels. That is, a particular parameter does not necessarily have the same reference value for all channels.

As the above examples concern ECG measurement, the apparatus/system is discussed in terms of the ECG measurement in this context. However, it is to be noted that no real ECG electrode placement is shown in FIG. 8. For the ECG measurement, the control and processing unit may be provided with a separate ECG measurement algorithm 805 adapted to acquire the ECG lead signal data from the subject. For the determination of the ECG related parameters, the control and processing unit may further be provided with one or more ECG parameter algorithms 806 adapted to calculate the ECG related parameters. Algorithms 805 or 806 may be provided with the filtering function of step 21 of FIG. 2. The control and processing unit may further be provided with an evolution measure algorithm 807 adapted to calculate the evolution measures, with a mapping algorithm 808 adapted to map the channel-specific evolution measures to plot colors, and with a presentation algorithm 809 adapted to control a user output device, such as a display unit 810, to present the evolution map and the desired signal waveforms to a user of the apparatus.

Consequently, in terms of the disclosed ECG monitoring process, the functionalities of the control and processing unit 803 may be divided into the units shown in FIG. 9. A measurement unit 90 is configured to acquire the lead signal data and a parameter determination unit 91 is adapted to determine one or more parameters for each measurement channel. An evolution determination unit 92 is configured to analyze the parameters and to determine the evolution measures, a mapping unit 93 is configured to map the evolution measures to plot colors, and a presentation unit 94 is configured to control a user output device, such as a display unit, to display the evolution plot and possibly also the waveforms. As discussed below, units 91 and 92 may also be combined to a single unit if the parameter value is used as such as the evolution measure. The mapping unit may use various mechanisms to associate the evolution measure value with a particular color and the resulting color may include a set of color attributes and may also be provided with display control data.

It is to be noted that FIGS. 8 and 9 illustrate the division of the functionalities of the control and processing unit in logical sense and in view of the visually informative ECG monitoring disclosed. In a real apparatus the functional ities may be distributed in different ways between the elements or units of the apparatus.

In the above examples, the evolution measure is mapped to a plot color similarly for all channels and signal segments. However, the color coding may also change in the middle of the monitoring process, for example to improve the resolution of the monitoring process. The color coding used at each moment may be displayed to the user on the screen of the display unit. Furthermore, different physiological parameters may have different severity weights. That is, a change in one parameter may be regarded more severe than a change in another parameter.

In a basic embodiment, the evolution plot may be displayed to the user without channel waveforms 51. In further embodiments, channel waveforms may be displayed in connection with the respective plot zones assigned to the channel, as is shown in FIGS. 5 to 7. All channel/lead signals may be presented in the same evolution plot or the channel signals may be divided between two or more evolution plots, which may displayed at the same time or alternatively. The user may divide the channel/lead signals between the plots in a preferred manner. For example, the chest channels of a standard 12 lead ECG may be displayed on one evolution plot and the limb and augmented limb channels on another evolution plot.

The monitoring of a subject may also be carried out so that the evolution plot(s) is/are displayed only if the ECG starts to deviate from normal ECG.

A conventional patient monitor may also be upgraded to show, in addition to the conventional waveform presentation, one or more evolution plots. Such an upgrade may be implemented, for example, by delivering to the monitor a plug-in unit that may be provided with the necessary software portions for enabling the control and processing unit to generate the color coded evolution plot based on the channel data. When color coded evolution plot is taken into use, the control and processing unit 803 executes the software portions to display the evolution plot to the user of apparatus/system. These software portions may correspond to the operational units 91-94 of FIG. 9, for example. However, the contents of the software portions may vary depending on the existing algorithms of the apparatus. For example, the conventional monitor may provide the physiological parameters needed for the evolution plot. The plug-in unit may be delivered, for example, on a data carrier, such as a CD or a memory card, or the through a telecommunications network. The apparatus may also be implemented as a separate display unit that comprises operational units 92-94 and receives the parameters from the conventional patient monitor.

As discussed above, various physiological parameters may be derived from the channel/lead signals. In addition to the features related to the repolarization phase, the duration of the QRS wave may be determined. Furthermore, instead of T wave amplitude T wave shape may be used to indicate a T wave change that may predict upcoming events, such as infarction. The method/apparatus may also be used in different configurations according to user preferences. For example, one user may like to measure T wave amplitude, while another prefers to find out changes in QRS duration. Thus, the user may choose different parameters to evaluate the clinical state of the subject.

The determination of the evolution measure may be carried out in different ways depending, for example, on how many parameters are determined for each channel. As mentioned above, the evolution measure may be derived from the difference of the current and reference values if only one parameter is determined for each channel. In a further embodiment, a single parameter may be determined for each channel, but the number of parameters may be increased if the current parameter results in an evolution measure that indicates a significant change in the state of the subject.

There may also be sudden changes in the ECG morphology that may be due to abnormal conduction or another reason, such as movement of the subject. For example, the subject may have a sudden bundle branch block resulting in a significant change in QRS and T wave morphologies. A similar situation may be caused by movement of the subject. These events may be detected and filtered out in the filtering step 21, for example, so that they do not affect the evolution measure. Further, in case of a more permanent morphology change, parameter threshold(s) defining the normal state of the subject may be reset in order to adapt the evolution measurement to the changed morphology.

If several parameters are determined at the same time, the channel-specific evolution measure may be determined as an aggregate value which may be obtained, for example, by deriving the absolute differences of the current and reference values for each parameter and calculating the evolution measure as a weighted and/or normalized sum of the absolute differences. In one embodiment, the channel-specific evolution measure may be obtained based on the absolute difference that indicates the greatest change with respect to the normal state (reference value). Value ranges may be defined for the evolution measures and each evolution measure may be mapped to the plot color (or color shade) that corresponds to the value range on which the current value of the evolution measure resides. A typical parameter currently used for infarction monitoring is ST level. A clinically significant change in ST level is typically 2 mm. A change of 1-2 mm may be considered a medium change, while a change of 0.5 mm may be considered a minor change. In addition, changes in T wave amplitude may also be considered, and if other signs indicative of adverse evolution are detected at the same time, such as inverted T or increased Q waves, the overall severity may be increased. For example, if an ST level change of 0.5 mm occurs, it is indicated as a minor change in evolution plot if no other signs indicative of adverse evolution are detected at the same time. If at the same time an increased Q-wave and/or inverted T-wave is/are detected, it may be indicated as a medium or significant change.

Sometimes it may also be important to measure evolution compared to a physiological reference defined by general population. For example, normal ST level should be less than 1 mm and thus crossing of this level may be indicative of abnormal oxygenation of heart, which may be indicated by a color change in the evolution plot. Consequently, the user may choose the reference data that defines the normal state based on patient history data and/or general population data and the current parameter value may serve as the evolution measure. That is, in some embodiments the difference of the current and reference values needs not to be determined. In these embodiments, different colors may be assigned to different value ranges of a parameter, such as ST level, and the current parameter value may be mapped directly to the plot color that corresponds to the said value.

With reference to FIG. 8 again, the evolution plot data may be stored in the memory 804 of the apparatus or in a memory/database 812 of a network, such as a hospital LAN 813. The time scale of the evolution plot may be adjustable, so that a user may select a desired time period for review. The plot history data corresponding to the selected time period may be retrieved from the network memory 812 through a network interface 814 and a database server 815 and displayed in a user display window on the screen of the display unit 810. By searching for certain colors or color patterns from the evolution plot covering the desired time period, the user of the apparatus may examine whether a specific event has occurred earlier. The visualization of the ECG through the colored evolution plot provides a physician an efficient tool to quickly search for certain events from the ECG history of the subject, and to get an impression of the occurrence of abnormal events. It is also possible that the evolution plot is produced for the examination of the patient history, by viewing the plot off-line based on the lead signal data stored earlier in a memory or database.

Above, ECG is mainly used as an example of the physiological signal. However, the above mechanism may also be applied to pulse oximetry, for example, where the amplitude or variation in the amplitude or pulse rate of a pulse oximeter waveform may be used as the parameters that are indicative of evolution. Variation in the amplitude of a pulse oximeter signal may indicate a volume change in blood, while changes and variation in the pulse rate, especially compared to ECG, may indicate electromechanical dissociation. Another possible application area is respiration measurement. In general, respiration rate and depth of respiration are important parameters in patient care and may therefore be used as parameters indicative of the clinical state of a subject, similarly as ECG related parameters are used above.

This written description uses examples to disclose the invention, including the best mode, and also to enable any person skilled in the art to make and use the invention. The patentable scope of the invention is defined by the claims, and may include other examples that occur to those skilled in the art. Such other examples are intended to be within the scope of the claims if they have structural or operational elements that do not differ from the literal language of the claims, or if they have structural or operational elements with insubstantial differences from the literal language of the claims.

1. A method for monitoring clinical state of a subject, the method comprising:
determining an evolution measure for each of a plurality of physiological channel signals acquired from a subject, thereby to obtain a corresponding plurality of channel-specific evolution measures, wherein each channel-specific evolution measure is indicative of development in respective channel signal;
mapping each channel-specific evolution measure to a plot color, thereby to obtain a channel-specific plot color for each of the plurality of physiological channel signals,
wherein the determining and mapping are carried out in successive time windows, thereby to obtain a corresponding plurality of channel-specific plot color sequences; and
presenting the corresponding plurality of channel-specific plot color sequences to a user as an indication of clinical state of the subject.

2. The method according to claim 1, further comprising deriving at least one physiological parameter from each of the plurality of physiological channel signals acquired from a subject, wherein the determining comprises determining the evolution measure based on the at least one physiological parameter.

3. The method according to claim 2, wherein the deriving includes deriving the at least one physiological parameter from each of the plurality of physiological channel signals, in which the plurality of physiological channel signals are ECG lead signals.

4. The method according to claim 1, wherein the presenting includes presenting the corresponding plurality of channel-specific plot color sequences as an evolution plot in which each of the corresponding plurality of plot color sequences is assigned a channel-specific plot zone.

5. The method according to claim 2, further comprising storing reference data for the subject, wherein the reference data indicates normal state of the subject and comprises at least one normal value for each of the at least one physiological parameter.

6. The method according to claim 1, further comprising assigning multiple dedicated plot colors respectively to multiple consecutive value ranges of the evolution measure, wherein the mapping comprises mapping each channel-specific evolution measure to a dedicated plot color that corresponds to current value of the evolution measure, wherein the dedicated plot color is any of the multiple dedicated plot colors.

7. The method according to claim 6, wherein the assigning includes assigning a first color to a first value range of the evolution measure and different shades of a second color to other value ranges of the evolution measure.

8. The method according to claim 7, wherein the first value range corresponds to normal clinical state of the subject.

9. An apparatus for monitoring clinical state of a subject, the apparatus comprising:
an evolution determination unit configured to determine an evolution measure for each of a plurality of physiological channel signals acquired from a subject, thereby to obtain a corresponding plurality of channel-specific evolution measures, wherein each channel-specific evolution measure is indicative of signal development in respective channel signal;
a mapping unit configured to map each evolution measure to a plot color, thereby to obtain a channel-specific plot color for each of the plurality of physiological channel signals,
wherein the evolution determination unit and the mapping unit are configured to operate in successive time windows, thereby to obtain a corresponding plurality of channel-specific plot color sequences; and
a presentation unit configured to present the corresponding plurality of plot color sequences to a user as an indication of clinical state of the subject.

10. The apparatus according to claim 9, further comprising a parameter determination unit configured to derive at least one physiological parameter from each of the plurality of physiological channel signals, wherein the evolution determination unit is configured to determine the evolution measure based on the at least one physiological parameter.

11. The apparatus according to claim 9, wherein the plurality of physiological channel signals are ECG lead signals.

12. The apparatus according to claim 9, wherein the presentation unit is configured to present the corresponding plurality of channel-specific plot color sequences as an evolution plot in which each of the corresponding plurality of plot color sequences is assigned a channel-specific plot zone.

13. The apparatus according to claim 10, further comprising reference data for the subject, wherein the reference data indicates normal state of the subject and comprises at least one normal value for each of the at least one physiological parameter.

14. The apparatus according to claim 13, wherein the mapping unit is adapted to map each channel-specific evolution measure to a dedicated plot color that corresponds to current value of the evolution measure.

15. The apparatus according to claim 14, wherein the mapping unit is configured to assign a first color to a first value range of the evolution measure and different shades of a second color to other value ranges of the evolution measure.

16. The apparatus according to claim 15, wherein the first value range corresponds to normal clinical state of the subject.

17. A computer program product for monitoring clinical state of a subject, the computer program product comprising:
a first program product portion adapted to determine an evolution measure for each of a plurality of physiological channel signals acquired from a subject, thereby to obtain a corresponding plurality of channel-specific evolution measures, wherein each channel-specific evolution measure is indicative of signal development in respective channel signal;
a second program product portion adapted to map each evolution measure to a plot color, thereby to obtain a channel-specific plot color for each of the plurality of physiological channel signals,
wherein the first and second program product portions are adapted to operate in successive time windows, thereby to obtain a corresponding plurality of channel-specific plot color sequences; and
a third program product portion adapted to present the corresponding plurality of plot color sequences to a user as an indication of clinical state of the subject.
